(19) United States
(12) Patent Application Publication
(10) Pub. No.: US 2025/0354956 A1
(43) Pub. Date: Nov. 20, 2025

(54) ANALYSIS METHOD BY ELECTROPHORESIS

(71) Applicant: ACE BioAnalysis Inc., Fukushima (JP)

(72) Inventor: Kiyohito SHIMURA, Fukushima (JP)

(21) Appl. No.: 19/040,120

(22) Filed: Jan. 29, 2025

(30) Foreign Application Priority Data
May 15, 2024 (JP) 2024-079700

(51) Int. Cl. G01N 27/447; G01N 33/58; G01N 33/68

(52) U.S. Cl. CPC G01N 27/44726; G01N 27/44795; G01N 33/582; G01N 33/6803

(57) ABSTRACT

The present disclosure provides a method for analyzing proteins and polypeptides by electrophoresis using standards as internal standards. In one aspect, the present disclosure provides a method for measuring an analyte by electrophoresis, the method including the steps of preparing standards that do not contain tryptophan, subjecting a sample containing the analyte and the standards to electrophoresis simultaneously in the same separation field, detecting the analyte with an optical signal derived from tryptophan and detecting the standards with an optical signal of a wavelength different from that of the analyte, and measuring the analyte based on the optical signal. In one embodiment, the electrophoresis is capillary electrophoresis.

### BACKGROUND OF THE INVENTION

### Technical Field

The present disclosure relates to methods for analyzing an analyte of interest by electrophoresis.

### Technical Field

There are many types of biological molecules such as proteins, including those modified by post-translational modifications, and various analytical methods have been developed due to the complexity of their molecular structures. Electrophoretic analytical methods such as capillary electrophoresis are often used to analyze biological molecules such as proteins. Biological molecules are often used as pharmaceutical ingredients, such as antibody drugs, and electrophoretic analytical methods can be simple and useful not only for research but also for confirming the quality of products produced in the manufacture of pharmaceutical ingredients.

Although analysis by capillary electrophoresis allows for robust analysis, reproducibility of absolute detection times between experiments (runs) is not necessarily high, so it is beneficial to increase the versatility of the information obtained by electrophoretic analysis.

### NON-PATENT DOCUMENTS

1. Shimura K, Kasai K. Fluorescence-labeled peptides as isoelectric point (pI) markers in capillary isoelectric focusing with fluorescence detection. Electrophoresis. 1995;16(8):1479-1484.
2. Verbeck IV GF, Beale SC. Isoelectric point analysis of proteins and peptides by capillary isoelectric focusing with two-wavelength laser-induced fluorescence detection. J Microcolumn September 1999;11(10):708-715.
3. Shimura K, Wang Z, Matsumoto H, Kasai K. Synthetic oligopeptides as isoelectric point markers for capillary isoelectric focusing with ultraviolet absorption detection. Electrophoresis. 2000;21(3):603-610.
4. Shimura K, Wang Z, Matsumoto H, Kasai K. Accuracy in the determination of isoelectric points of some proteins and a peptide by capillary isoelectric focusing: utility of synthetic peptides as isoelectric point markers. Anal Chem. 2000;72(19):4747-4757.
5. Shimura K, Kamiya K, Matsumoto H, Kasai K. Fluorescence-labeled peptide pI markers for capillary isoelectric focusing. Anal Chem. 2002;74(5 00005):1046-1053.
6. Shimura K. Recent advances in capillary isoelectric focusing: 1997-2001. Electrophoresis. 2002;23(22-23):3847-3857.
7. Righetti PG. Determination of the isoelectric point of proteins by capillary isoelectric focusing. J Chromatogr A. 2004;1037(1-2 00249):491-499.
8. Wu J, Huang T. Peak identification in capillary isoelectric focusing using the concept of relative peak position as determined by two isoelectric point markers. Electrophoresis. 2006;27(18 00586):3584-3590.
9. Shimura K. Recent advances in IEF in capillary tubes and microchips. Electrophoresis. 2009;30(1):11-28.
10. Shimura K. Capillary Isoelectric Focusing. In: Poole CF, ed. Capillary Electromigration Separation Methods. Elsevier; 2018:167-187.

### PATENT DOCUMENTS

### 1. J.P. No. 2828426, 9/1998, Shimura K et al.

### 2. U.S.P. No. 5866683, 2/1999, Shimura K et al.

### 3. J.P. No. 3148153, 1/2001, Matsumoto H et al.

### 4. J.P. No. 3246888, 11/2001, Matsumoto H et al.

### 5. J.P. No. 3520222, 2/2004, Matsumoto H et al.

### SUMMARY OF THE INVENTION

As a result of intensive research, the present inventor has developed a method for improving electrophoretic analysis by using standard substances (markers). Based on this, the present disclosure provides an electrophoretic analysis method using standard substances, standard substances therefor, an apparatus therefor, and the like.

Thus, the present disclosure provides the following:

### Item 1

A method for measuring an unlabeled analyte, which is a protein and/or polypeptide, by electrophoresis, comprising the steps of:
subjecting a sample containing the analyte and standards to electrophoresis simultaneously in the same separation space;
detecting the analyte with an optical signal derived from tryptophan and detecting the standard with an optical signal of a different wavelength from the analyte;
and measuring the analyte based on the optical signal.

### Item 2

The method of item 1 for determining the isoelectric point of an analyte, wherein the standards have known isoelectric points.

### Item 3

The method of item 2, wherein the standards comprise a plurality of standards having different known isoelectric points ranging from 2.5 to 11.5.

### Item 4

The method of item 1 for determining the molecular weight of an analyte, wherein the standards have known molecular weights.

### Item 5

The method of item 4, wherein the standards comprise a plurality of standards having different known molecular weights ranging from 5 kDa to 1000 kDa.

### Item 6

The method according to item 1, further comprising deriving an equation expressing a correlation between the isoelectric point and the detection time or detection position, or between the molecular weight and the detection time or detection position, based on optical signals derived from the plurality of standard substances.

### Item 7

The method of item 6, further comprising converting the detection time or detection position into a pH value or a molecular weight based on said equation.

### Item 8

The method of item 1, wherein the standard is a peptide or protein that does not contain tryptophan.

### Item 9

The method of item 1, wherein the standard is a dye-labeled peptide or protein.

### Item 10

The method of item 1, wherein the standard has a chromophore, fluorophore, or dye label.

### Item 11

The method of item 1, wherein the step of detecting an optical signal comprises irradiating with light having two wavelengths, a wavelength of about 280 nm and a wavelength suitable for detecting the chromophore, fluorophore or dye label of the standard.

### Item 12

The method of item 1, wherein the standard is labeled with rhodamine.

### Item 13

The method of item 1, wherein the step of detecting the optical signal comprises illuminating with light having a single wavelength.

### Item 14

The method of item 13, wherein the single wavelength is about 280 nm.

### Item 15

The method of item 1, wherein a plurality of standards is prepared so as to be detected as peaks of distinguishable different magnitudes.

### Item 16

The method of item 1, wherein the electrophoresis is capillary electrophoresis.

### Item 17

An apparatus for measuring an analyte by electrophoresis, comprising:
a flow path and electrodes for electrophoresis;
a light source for illuminating at least a portion of the flow path;
and a photodetector for receiving light from the flow path,
wherein the flow path has an inlet for receiving a sample containing the analyte.

### Item 18

The apparatus of item 17, wherein the light source is configured to illuminate the flow path with light of a single wavelength.

### Item 19

The apparatus of item 17, wherein the light source is configured to illuminate the flow path with light having a wavelength of about 280 nm.

### Item 20

The apparatus of item 17, wherein the photodetector is configured to detect light of a different wavelength than the illumination light of the light source.

### Item 21

The apparatus of item 17, wherein the photodetector is configured to detect light at two wavelengths.

### Item 22

The apparatus of item 21, wherein the photodetector comprises a dichroic mirror, a dichroic filter, or a beam splitter.

### Item 23

The apparatus of item 21, wherein the photodetector is configured to detect light having a wavelength of about 350 nm and light having a wavelength of about 575 nm.

### Item 24

A composition for determining the isoelectric point of an analyte, comprising a plurality of standards,
wherein the composition is intended to be subjected to isoelectric focusing in the same separation space simultaneously with a sample containing the analyte,
wherein the standards have known isoelectric points that are different from one another.

### Item 25

A composition for determining the molecular weight of an analyte, comprising a plurality of standards,
wherein the composition is intended to be subjected to electrophoresis in the same separation space simultaneously with a sample containing the analyte,
wherein the standards have known molecular weights that are different from one another.

### Item 26

A method for producing a standard substance for determining the molecular weight of an analyte, comprising the steps of:
identifying a gene encoding a protein having a known molecular weight;
identifying the base sequence encoding all tryptophan residues of the protein in the gene;
obtaining a modified gene by replacing the portion encoding all tryptophan residues with a base sequence encoding amino acids other than tryptophan;
obtaining a protein produced using the modified gene;
and obtaining a conjugate formed by binding a labeling dye to the protein as the standard substance.

It is contemplated that one or more of the above features may be provided in combinations other than those explicitly stated. Still further embodiments and advantages of the present disclosure will be recognized by those skilled in the art upon reading and understanding the following detailed description, if necessary.

The present disclosure provides a simple electrophoretic analysis using standard substances as internal standards.

### BRIEF DESCRIPTION OF THE DRAWINGS

FIG. 1 is a schematic diagram of an exemplary fluorescence detector for capillary electrophoresis.

FIG. 2 shows the results of separation of cetuximab, edge markers (EM) and flanking markers (FM) (Table 1) by capillary isoelectric focusing (CIEF). 33 ng of cetuximab and 0.3 pmol each of four tryptophan-containing peptide pI markers (EM3.38, FM7.00, FM9.50, EM10.17) were separated by CIEF using a carrier ampholyte that generates a pH gradient from pH 3 to 10.

The results of the eighth scanning detection after the start of focusing at 10 kV are shown, corresponding 12 min of focusing time at 10 kV. The solid line indicates the fluorescence at 340 nm derived from tryptophan residues. EM: edge markers, FM: flanking markers, C: cetuximab. The horizontal axis indicates the detection time proportional to the axial position of the capillary, and the vertical axis indicates the value of the fluorescence signal proportional to the amount of cetuximab and markers.

FIG. 3 shows the results of separation of cetuximab and twelve ubiquitous markers (UM) (Table 1) by CIEF. 33 ng of cetuximab and 8 fmol each of twelve tetramethylrhodamine-labeled peptide pI markers (UM7.58, UM8.21, UM8.77, UM9.56, etc.) were separated by CIEF using a carrier ampholyte that generates a pH gradient from pH 3 to 10. The results are from the eighth scan detection after the start of focusing at 10 kV. The solid line shows the fluorescence at 340 nm derived from tryptophan residues in cetuximab, and the dotted line shows the fluorescence at 575 nm of tetramethylrhodamine label of markers. UM: ubiquitous markers, C: cetuximab. The horizontal axis indicates the detection time, and the vertical axis shows the value of the fluorescence signal.

FIG. 4 shows a plot of the separation result of cetuximab shown in FIG. 3 on a pH axis. The time axis in FIG. 3 was converted to a pH axis based on the isoelectric point and detection time of each ubiquitous marker.

FIG. 5 shows the isoelectric points of the cetuximab peaks (C3 to C9) and their changes under focusing conditions that were determined using edge markers, flanking markers, and ubiquitous markers, respectively. A) Using edge markers (markers with pI 3.38 and pI 10.17), B) Using flanking markers (markers with pI 7.00 and pI 9.50), C) Using ubiquitous markers (markers with pI 7.58, pI 8.21, pI 8.77, and pI 9.56). The correspondence between the symbols and the cetuximab peaks is shown at the bottom of FIG. 5. C3 to C9: peaks of cetuximab as shown in FIGS. 2 and 3.

FIG. 6 shows the results of separation of ubiquitous markers by CIEF. The horizontal axis indicates the detection time, and the vertical axis indicates the value of the fluorescence signal.

FIG. 7 shows the approximation by a third-order polynomial of the pH value versus detection time for the separation results in FIG. 6, and its graph. The horizontal axis is the detection time (min), and the vertical axis is the pH value of the pH gradient.

### DETAILED DESCRIPTION OF THE INVENTION

Hereinafter, the present disclosure will be described with reference to the best mode. Throughout this specification, the singular expression should be understood to include the concept of the plural, unless otherwise specified. Thus, the article used in the singular (e.g., in the case of English, “a”, “an”, “the”, etc.) should be understood to include the concept of the plural, unless otherwise specified. In addition, it should be understood that the terms used in this specification are used in the sense commonly used in the field, unless otherwise specified. Thus, unless otherwise defined, all technical terms and scientific terms used in this specification have the same meaning as commonly understood by those skilled in the art to which this disclosure belongs. In the case of conflict, this specification (including definitions) will take precedence.

The following provides definitions of terms particularly used in this specification and/or basic technical content as appropriate.

### Definitions, Etc.

As used herein, “electrophoresis” refers to the operation of generating a potential difference between two separate points in a channel filled with a solution of an electrolyte, causing the movement of a charged substance present between the two points. When electrophoresis is performed using a capillary tube (a tube with a hollow structure having a small inner diameter, typically about 0.01 to about 1 mm), it can be described as capillary electrophoresis.

As used herein, the term “isoelectric focusing” refers to electrophoresis that performs separation in a pH gradient by utilizing the phenomenon in which the net charge of an ampholyte becomes zero at a certain pH value (isoelectric point) and no longer migrates by electrophoresis.

In this specification, the term “analyte” refers to a substance to be separated and analyzed. Usually, the properties of the analyte to be analyzed are unknown. It may be unknown whether the analyte is present in the sample, so a sample containing the analyte described in this specification also refers to a sample that is expected to contain the analyte, and the measurement may result in the determination that the analyte is not contained. Typically, the analyte described in this specification is a protein (or peptide) including glycoproteins, lipoproteins, metal proteins, etc.

As used herein, proteins include peptides, but generally, the term “protein” refers to a molecule having a relatively large molecular weight, and the term “peptide” refers to a molecule having a relatively small molecular weight (e.g., 30 amino acids or less, 20 amino acids or less, 15 amino acids or less). The biological molecules described herein also include molecules containing modifications such as phosphorylation, glycosylation, and sialylation in proteins, as well as molecules conjugated with a label such as a fluorescent label.

As used herein, the term “standard” (sometimes referred to as “marker”) refers to a substance having a known property to be measured (isoelectric point, molecular weight, etc.). Typically, the standard described herein is an internal standard, which is mixed with the analyte and detected in the same measurement (run).

As used herein, the term “kit” refers to a unit in which the parts that can be provided separately are provided as a set for the convenience of users. The kit preferably advantageously includes an instruction manual or manual that describes how to use or operate the parts provided.

As used herein, the term “about” refers to a range of the indicated value plus or minus 10%, unless otherwise specified. When “about” is used in reference to temperature, it refers to a range of the indicated temperature plus or minus 5° C.

### Preferred Embodiment

The preferred embodiment of the present disclosure will be described below. The embodiments provided below are provided for a better understanding of the present disclosure, and it is understood that the scope of the present disclosure should not be limited to the following description. Therefore, it is clear that a person skilled in the art can make appropriate modifications within the scope of the present disclosure in consideration of the description in this specification. It is also understood that the following embodiments of the present disclosure can be used alone or in combination.

In one aspect, the present disclosure provides a method for measuring an analyte by electrophoresis, comprising the steps of: mixing a sample containing the analyte with standards to prepare a mixture; subjecting the mixture to electrophoresis; detecting optical signals derived from the analyte and the standards; and measuring the analyte based on the optical signals. In one embodiment, the electrophoresis is capillary electrophoresis or microfluidic chip electrophoresis.

In one embodiment, the isoelectric point of the analyte is determined by electrophoresis. In this embodiment, the standards have known isoelectric point, and the electrophoresis is isoelectric focusing or the like. In one embodiment, the molecular weight of the analyte is determined by electrophoresis. In this embodiment, the standards have known molecular weight, and the electrophoresis is gel electrophoresis, polymer solution electrophoresis or the like.

### Standard Substance

The standard substance is any substance capable of electrophoresis. In an electrophoretic analysis method, the substance is easily detected by light, so the standard substance preferably has an optical characteristic group (fluorescent group, light absorbing group, chromogenic group) that allows optical detection.
In one embodiment, the standard substance is a protein (peptide).

In many embodiments of the present disclosure, the analyte is a protein (peptide), and the standard is preferably detected in a manner that is distinguishable from the analyte. In particular, when the standard is detected at a detection time close to that of the analyte in electrophoresis, it is preferable that the standard and the analyte can be distinguished based on the difference in optical properties. In one embodiment, the standard is a protein (peptide) that does not contain tryptophan. Since proteins (peptides) usually contain tryptophan, the optical properties of tryptophan, namely, an excitation (absorption) spectrum having a peak at about 280 nm and a fluorescence (emission) spectrum having a peak at about 350 nm, can be used to optically detect the protein (peptide). Therefore, by designing the tryptophan-free standard to include an optical property group (such as a fluorescent dye label) that can be detected at a wavelength different from that used to detect tryptophan, the protein (peptide) analyte can be detected independently of the standard. For example, examples of optical groups having fluorescence at a wavelength different from that of tryptophan include rhodamine, fluorescein, cyanine, indocyanine, indocarbocyanine, pyronine, lucifer yellow, quinacrine, squaric acid, coumarin, and fluoranthene. For example, optical groups having an absorption peak at a wavelength different from that of tryptophan include, but are not limited to, the same optical groups as those having fluorescence at a wavelength different from that of tryptophan. Those skilled in the art can appropriately perform electrophoretic analysis while considering the influence of electrophoretic conditions (e.g., electrophoresis medium) on optical properties.

Similarly, when the analyte is a nucleic acid, the standard can be designed to contain an optical group that is detectable at a wavelength different from that of adenine, guanine, thymine, cytosine, and uracil, making it possible to distinguish the analyte from the standard.

In one embodiment, the standard comprises a plurality of standards (e.g., 2, 3, 4, 5, 7, 10, 15, 20 or more). In a preferred embodiment, the plurality of standards comprises a common optical group (or different optical groups having a common emission, fluorescence or absorption wavelength) so that they can be detected together. In a preferred embodiment, the plurality of standards has different known isoelectric points or known molecular weights.

Standards can be selected appropriately depending on the purpose of the analysis. For example, if the analyte is completely unknown, multiple standards with a wide range of isoelectric points or molecular weights can be used.
For example, if the analyte is approximately identified, such as a pharmaceutical ingredient, two or more standards with isoelectric points or molecular weights close to the isoelectric point or molecular weight of interest can be used.

The amount of standard to be used can be appropriately determined by a person skilled in the art so that the standard can be detected. In one embodiment, the concentration of the standard in the mixture obtained by mixing with the sample may be about 0.1 nM to 100 μM, e.g., about 1 nM to 100 μM, about 10 nM to 100 μM, about 100 nM to 100 μM, about 1 μM to 100 μM, about 0.1 nM to 10 μM, about 1 nM to 10 μM, about 10 nM to 10 μM, about 100 nM to 10 μM, about 1 μM to 10 μM, about 0.1 nM to 1 μM, about 1 nM to 1 μM, about 10 nM to 1 μM, about 100 nM to 1 μM, about 0.1 nM to 100 nM, about 1 nM to 100 nM, about 10 nM to 100 nM, about 0.1 nM to 10 nM, or about 1 nM to 10 nM. Even when irradiating the standard at a wavelength (e.g., about 280 nm) that is not the optimal excitation wavelength for the standard (e.g., about 520-550 nm for rhodamine), a standard at a concentration of about 10 nM may be detectable. In one embodiment, multiple standards are mixed with the sample at different concentrations, so that the magnitude of the detected peaks differs for each standard, making it easier to identify the standard peaks.

In one embodiment, the standards include multiple standards having different known isoelectric points within the range of about 1 to 13, about 2 to 12, or about 3 to 11. In one embodiment, the standards for isoelectric point measurement have a difference in isoelectric points between adjacent standards of less than about 4, less than about 3.5, less than about 3, less than about 2.5, less than about 2, less than about 1.5, or less than about 1.

In one embodiment, the present invention includes a plurality of standards having different known molecular weights ranging from about 1 kDa to 100 MDa, e.g., from about 1 kDa to 10 MDa, from about 1 kDa to 5 MDa, from about 1 kDa to 2 MDa, from about 1 kDa to 1 MDa, from about 1 kDa to 500 kDa, from about 1 kDa to 200 kDa, from about 2 kDa to 100 MDa, from about 2 kDa to 10 MDa, from about 2 kDa to 5 MDa, from about 2 kDa to 2 MDa, from about 2 kDa to 1 MDa, from about 2 kDa to 500 kDa, from about 2 kDa to 200 kDa, from about 5 kDa to 100 MDa, from about 5 kDa to 10 MDa. from about 5 kDa to 5 MDa, about 5 kDa to 2 MDa, about 5 kDa to 1 MDa, about 5 kDa to 500 kDa, about 10 kDa to 100 MDa, about 10 kDa to 10 MDa, about 10 kDa to 5 MDa, about 10 kDa to 2 MDa, about 10 kDa to 1 MDa, about 20 kDa to 100 MDa, about 20 kDa to 10 MDa, about 20 kDa to 5 MDa, about 20 kDa to 2 MDa, or about 20 kDa to 1 MDa. In one embodiment, the difference in molecular weight between adjacent standards for the measurement of molecular weight is about 1.1-1000 times, e.g., about 1.1-500 times, about 1.1-200 times, about 1.1-100 times, about 1.1-50 times, about 1.1-20 times, about 1.1-10 times, about 5-1000 times, about 5-500 times, about 5-200 times, about 5-100 times, about 5-50 times, about 5-20 times, about 10-1000 times, about 10-500 times, about 10-200 times, about 10-100 times, about 10-50 times, about 20-1000 times, about 20-500 times, about 20-200 times, about 20-100 times, about 50-1000 times, about 50-500 times, or about 50-200 times.

A standard substance having a specific isoelectric point or molecular weight can be appropriately prepared by a person skilled in the art. A protein or peptide standard substance having a specific isoelectric point or molecular weight can be obtained using any known recombinant gene expression technology, peptide synthesis technology, and peptide modification technology.

It is also possible to estimate the isoelectric point of a peptide and design a peptide standard substance with a specific isoelectric point. The total charge (Z) of a peptide can be estimated by the following equation (Kagaku no ryoiki, 36, 470-486, 1982):

$$Z = \sum i ( ni / ( 1 + Ki / [ {H}^{+} ] ) ) - \sum j ( nj / ( 1 + [ {H}^{+} ] / Kj ) ) .$$

where Ki, is the acid dissociation constant (Ka) for the conjugate acid of the basic group and Kj is that for the acidic group forming a negatively charged conjugate base; ni and nj denote the numbers of such ionizable groups in a particular peptide. The value of pH=−log[H⁺] that makes Z=0 is the isoelectric point (pI) of the peptide, and it shows prediction of the isoelectric point of the peptide is possible. For example, the pKa of the functional groups of amino acids can be used to estimate the pI of a peptide, and examples include a-carboxyl (C-terminus) (3.6), β-carboxyl (Asp) (3.95), γ-carboxyl (Glu) (4.45), imidazole (His) (6.45), α-amino (N-terminus) (7.6), thiol (Cys) (8.5), phenolic hydroxyl (Tyr) (9.8), ε-amino (Lys) (10.2), and guanidinium group (Arg) (12.5). The polypeptides designed in this way can be synthesized and the isoelectric point can be experimentally determined.

Protein standards having a specific molecular weight can be easily obtained by purifying proteins (such as albumin) that are produced in large quantities in living organisms, but they may also be synthesized in vitro or modified by cleavage (e.g., cleavage using an enzyme such as trypsin) or fusion. Tryptophan-free proteins can be easily produced by those skilled in the art by identifying a gene that codes for a protein of interest, modifying the gene to remove the base sequence that codes for tryptophan, cloning the modified gene, and introducing it into a protein-producing cell. Assuming that the frequency of tryptophan among the amino acids that make up a protein (or peptide) is 1.3% and that the average molecular weight of one amino acid residue is 100 Da, the probability that a 50 kDa protein (or peptide) does not contain any tryptophan is about 0.14%, and the probability that a 100 kDa protein (or peptide) does not contain any tryptophan is about 0.00021%.

In one embodiment, the present disclosure provides a method for producing a standard for determining the molecular weight of an analyte, the method comprising the steps of identifying a gene encoding a protein having a known molecular weight, identifying a portion of the gene that encodes all tryptophans in the base sequence encoding the protein, replacing the portion that encodes all tryptophans with a base sequence encoding amino acids other than tryptophan to obtain a modified gene, and obtaining a protein by expression of the modified gene as a standard.

Methods for introducing labels (e.g., fluorescent dye labels) into proteins (or peptides) are known to those skilled in the art, and include, for example, a method of using a labeled amino acid as an amino acid raw material in in vitro peptide synthesis, a method of contacting a label with a reactive group (e.g., a carboxyl group converted to NHS) with a protein (or peptide) to chemically conjugate the label. For the purposes of this disclosure, the location of the label introduction does not significantly affect the analysis results. A standard substance having a specific isoelectric point can be purified after label introduction to obtain a standard substance having a uniform isoelectric point. For a standard substance having a specific molecular weight, the amount of change in molecular weight due to label introduction can be negligible in analysis. In addition, when labeling is performed by specifying the number of the label introduced, the change in molecular weight due to labeling can be calculated. This can be done by introducing free (non-cystine-forming) cysteine residues into a protein or peptide and labeling with a labeling dye having an iodoacetyl group or maleimide group that specifically reacts with SH group of cysteine.

### Light Detection

In one embodiment, in the step of subjecting the sample to electrophoresis and/or the step of detecting an optical signal of the present disclosure, light may be irradiated onto a flow path (such as a capillary) through which the sample and standard substance flow. The irradiated light may be absorbed by the analyte and/or standard substance, and this light absorption may be detected, or the irradiated light may excite chemical groups contained in the analyte and/or standard substance, resulting in the emission of fluorescence.

In one embodiment, the irradiated light may be limited to a specific wavelength or may include light of a specific wavelength. The wavelength of the light can be adjusted using an optical filter or the like, and light of a wavelength width of, for example, a specific wavelength±about 5 nm, 10 nm, about 20 nm, or about 30 nm can be generated. In this specification, unless otherwise specified, a specific wavelength refers to light having a maximum wavelength peak within about ±30 nm of that value. Since proteins (polypeptides) usually contain tryptophan, an analyte that is a protein (polypeptide) can be detected by irradiating light of a wavelength of about 280 nm suitable for exciting tryptophan and detecting the emitted light of a wavelength of about 350 nm. Since the fluorescence yield of tryptophan may be low compared to groups designed as fluorophores, it is preferable that the irradiation wavelength includes about 280 nm suitable for exciting tryptophan. In one embodiment, light of a second wavelength suitable for exciting the fluorophore of the standard is further irradiated. In one embodiment, one type of light having a maximum wavelength peak within about ±30 nm of the excitation maximum wavelength of the analyte is irradiated. The analyte is preferably detected with high sensitivity using a suitable excitation wavelength, but the standard can still be detected using a wavelength outside the range of suitable excitation wavelengths by increasing the concentration of the standard, etc., so the benefit of reducing the number of excitation light sources may outweigh the drawback of lower detection sensitivity. Since the analyte and the standard can preferably be detected in different types of light, the standard may have a fluorescent dye label that emits fluorescence with a maximum wavelength that is about 50 nm or more away from the maximum wavelength of the fluorescence from the analyte.

The light source is not particularly limited, but a laser that has high wavelength purity and enables stable light irradiation is preferable. For example, an argon laser, a semiconductor-pumped YAG laser, a helium-neon laser, etc. can be used.

### Determination of Isoelectric Point

The method disclosed herein can determine an accurate isoelectric point by using a standard substance as an internal standard. In one embodiment, an equation expressing the correlation between isoelectric point and detection time is obtained based on optical signals derived from multiple standards, and the detection time is converted to a pH value based on this equation, thereby determining an accurate isoelectric point. The equation may be a linear approximation equation based on a first-order approximation, but accuracy can be improved by approximating using a higher-order polynomial or the like. It is also effective to convert the detection position or time to pH, assuming that there is a proportional relationship between the isoelectric points (pH) of two adjacent standards and the positions or times at which the peaks of the two standards are detected. In this case, a pH gradient can be depicted for n standards by n−1 straight lines connected in series.

By using multiple standards, it is possible to provide standards with closer isoelectric points for many types of analytes with wide range of isoelectric points, and by providing reference isoelectric points using many standards, the accuracy of the approximation formula can be improved, resulting in precise determination of the isoelectric point. The isoelectric point determined with high precision becomes valuable information that can be commonly used by multiple practitioners working with the same analytes, regardless of laboratory conditions. It can also be effectively used to identify peaks found in other samples. Similarly, for molecular weight, an equation expressing the correlation between molecular weight and detection time can be derived based on optical signals derived from multiple standards, and the detection time can be converted to molecular weight based on this equation, allowing precise determination of the molecular weight.

### Composition, Kit

In one aspect, the present disclosure provides a composition or kit comprising a standard for measuring an analyte by electrophoresis. The composition or kit can be used in the methods described herein. In one embodiment, the composition or kit comprises a plurality of standards having different known isoelectric points, and the compositions or standards are mixed with a sample containing an analyte and then subjected to isoelectric focusing. In one embodiment, the composition or kit comprises a plurality of standards having different known molecular weights, and the compositions or standards are mixed with a sample containing an analyte and then subjected to electrophoresis.

### Apparatus

In one aspect, the present disclosure provides an apparatus for measuring an analyte by electrophoresis, the apparatus comprising a flow path and electrodes for electrophoresis, a light source for illuminating at least a portion of the flow path, and a photodetector for receiving light from the flow path, the flow path comprising an inlet for receiving a sample containing the analyte.

In one embodiment, the light source is configured to illuminate the flow path with light of a single wavelength. In one embodiment, the light source is configured to detect light of two wavelengths. In one embodiment, the light source is configured to illuminate the flow path with light of a wavelength of about 280 nm. For example, the light source can include an optical filter to adjust the wavelength of the light. The device may include a collector, such as a collimator.

In one embodiment, the photodetector is configured to detect light of a wavelength different from the illumination light of the light source. In one embodiment, the photodetector is configured to detect light of a wavelength of about 350 nm and/or light of a wavelength of about 575 nm. In one embodiment, the photodetector comprises a dichroic mirror, a dichroic filter, or a beam splitter.

In one embodiment, the flow path is a capillary. Capillaries can be commercially available, for example, as tubing for capillary electrophoresis or HPLC. The capillary can have an outer diameter of, for example, about 0.05-5 mm, about 0.05 mm, about 0.1 mm, about 0.18 mm, about 0.2 mm, about 0.36 mm, about 0.5 mm, about 1 mm, about 2 mm, about 5 mm, etc. The capillary can have an inner diameter of, for example, about 0.01-1 mm, about 0.01 mm, about 0.02 mm, about 0.05 mm, about 0.1 mm, about 0.2 mm, about 0.5 mm, about 1 mm, etc. The capillary can be made of, for example, fused silica or glass. The capillary may be coated on the outside with polyimide or the like.

### Note

In this specification, “or” is used when “at least one or more” of the items listed in the text can be adopted. In this specification, when it is specified that “within the range” of “two values,” the range includes the two values themselves.

The present disclosure will be described below based on examples, but the above description and the following examples are provided for illustrative purposes only and are not intended to limit the present invention. Therefore, the scope of the present invention is not limited to the embodiments or examples specifically described in this specification but is limited only by the claims.

### EXAMPLES

### Example 1: Determining Isoelectric Point Using Internal Standard Markers

A method that can determine the isoelectric point with high precision and easily was investigated by using pI markers as an internal standard.

### 1.1 Capillary Isoelectric Focusing (CIEF)

The antibody drug cetuximab was used as an analyte and separation by CIEF was performed using a scanning-detection capillary isoelectric focusing instrument (manufactured by Nichiei Kogyo Co., Ltd., Fukushima City). The capillary was a fused silica capillary with an inner diameter of 50 μm, an outer diameter of 375 μm, and a length of 420 mm, and the inner wall was coated with a hydrophilic polymer. A segment of polyimide coating on the outer wall of the capillary was removed to enable scanning detection by fluorescence. A contactless electric-conductivity detector was installed at a position 156 mm from the cathode end of the capillary. The carrier ampholyte solution used for separation after mixing with the sample consisted of 2.5% (v/v) Pharmalyte 3-10, 0.1% (v/v) acetic acid, and 0.35% (v/v) tetramethylethylenediamine. The anolyte was 0.1M phosphoric acid, and the catholyte was 1M sodium hydroxide. The protein analyte solution and a solution of standard substances with known isoelectric points (pI markers) were added to the carrier ampholyte solution to prepare a CIEF separation mix. The CIEF separation mix was injected from the anode side at 50 kPa for 1 minute into a capillary, and then the anolyte was injected from the anode side at 50 kPa until an increase in electric conductivity due to the anolyte was detected. Next, the capillary ends were immersed in the anolyte and catholyte, and after applying current at 5 kV for 1.5 minutes and then at 7 kV for 1.5 minutes, focusing was performed at 10 kV. During focusing at 10 kV, the range from 115 mm to 35 mm from the cathode end was scanned and detected from the anode side at a speed of 1 mm/s once every 1.5 minutes.

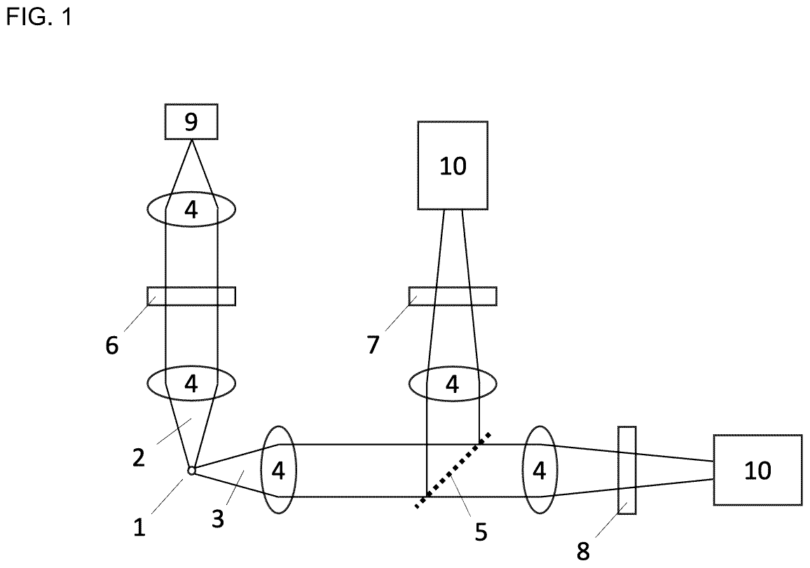

The schematic of the fluorescence detector is shown in FIG. 1. A 280 nm LED was used as the excitation light source for fluorescence detection, and the capillary was irradiated through a 280 nm bandpass filter. The collected fluorescence was divided using a beam splitter: 70% of the fluorescence was passed through a 340 nm bandpass filter and detected by a photomultiplier tube, and 30% of this was passed through a 575 nm bandpass filter and detected by another photomultiplier tube. The 340 nm fluorescence is derived from tryptophan (Trp) contained in analyte proteins and pI markers containing tryptophan, and the 575 nm fluorescence is derived from tetramethylrhodamine, a fluorescent dye contained in tryptophan-free pI markers.

### 1.2 The pI Markers for CIEF Separation Mix

Four pI markers containing tryptophan and twelve tryptophan-free pI markers labeled with tetramethylrhodamine were used. These pI markers are shown in Table 1.

TABLE 1
The way of use, structure and isoelectric point (pI)
of pI markers used for CIEF analysis of cetuximab

| Way of use | Structure | pI |
|---|---|---|
| Edge | H-Trp-Asp-Asp-Asp-OH | 3.38 |
| Flanking | H-Trp-Glu-His-Arg-OH | 7.00 |
| Flanking | H-Trp-Tyr-Tyr-Tyr-Lys-Lys-OH | 9.50 |
| Edge | H-Trp-Tyr-Lys-Arg-OH | 10.17 |
| Ubiquitous | H-Gly-Cys*-Asp-Asp-Asp-OH | 3.64 |
| Ubiquitous | H-Gly-Cys*-Glu-Glu-OH | 3.99 |
| Ubiquitous | H-Gly-Cys*-Glu-Glu-His-OH | 4.50 |
| Ubiquitous | H-Gly-Cys*-Glu-His-Glu-His-Glu-His-Glu-Lys-Glu-OH | 4.99 |
| Ubiquitous | H-Gly-Cys*-His-Glu-His-Glu-His-Glu-His-Glu-OH | 5.53 |
| Ubiquitous | H-Gly-Cys*-His-Glu-His-Glu-His-Glu-His-OH | 6.18 |
| Ubiquitous | H-Gly-Cys*-Glu-His-His-OH | 6.86 |
| Ubiquitous | H-Gly-Cys*-Glu-His-His-His-Arg-OH | 7.58 |
| Ubiquitous | H-Lys(H-Gly)-Cys*-Lys-Lys(H-Gly)-Glu-OH | 8.21 |
| Ubiquitous | H-Lys(H-Gly)-Cys*-Glu-Tyr-Tyr-Lys-Lys-Tyr-OH | 8.77 |
| Ubiquitous | H-Gly-Cys*-Tyr-Tyr-Tyr-Lys-Lys-OH | 9.56 |
| Ubiquitous | H-Gly-Cys*-Tyr-Lys-Arg-OH | 10.12 |

Note 1)
All amino acids except glycine are in the L-form.
Note 2)
Cys* indicates a cysteine residue whose SH group has been labeled with tetramethylrhodamine.
Note 3)
Lys(H-Gly) indicates a lysine residue whose epsilon-amino group is glycylated.

Of the four tryptophan-containing pI markers, two are located at almost both edges of the pH gradient spanning from pH 3 to 10, with pIs of 3.38 and 10.17. These are called edge markers (EM). The other two tryptophan-containing pI markers are located more closely on either side of the region in which the protein analyte cetuximab is distributed, with pIs of 7.00 and 9.50. These are called flanking markers (FM). The edge markers and flanking markers were used at a concentration of 1 μM for each in the CIEF separation mix that was injected to the capillary. The tetramethylrhodamine-labeled pI marker mix contains twelve markers with pIs of 3.64, 3.99, 4.50, 4.99, 5.53, 6.18, 6.86, 7.58, 8.21, 8.77, 9.56, and 10.12, and used each at a concentration of 20-30 nM in the CIEF separation mix. The twelve tetramethylrhodamine-labeled pI markers are called ubiquitous markers (UM) because they are dispersed throughout the pH gradient irrespective of the pI of analytes. The CIEF separation mix was prepared by adding 0.2 μL of cetuximab (5 mg/mL), 0.1 μL each of edge marker mix and flanking marker mix, or 0.1 μL of ubiquitous marker mix to 9 μL of the carrier ampholyte solution.

### 1.3 CIEF of Cetuximab and pI Markers

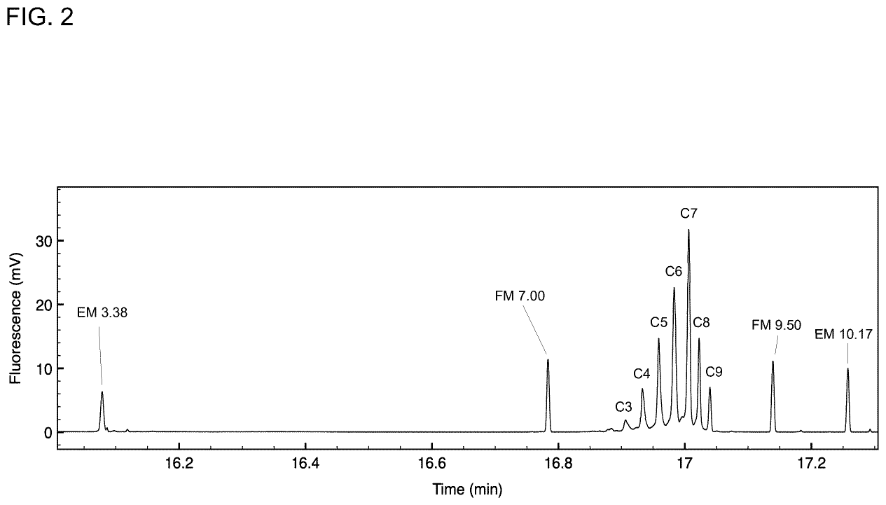

FIG. 2 shows the result of the eighth scan of separation of cetuximab (C3-C9) and edge markers (EM) and flanking markers (FM) by CIEF. These markers contain tryptophan, so they were detected simultaneously with cetuximab at a fluorescence wavelength of 340 nm. When determining the isoelectric point of an analyte by CIEF, it is often assumed that there is a proportional relationship between the detection time (or position) and the isoelectric point between the two isoelectric point markers present on both sides of the analyte, and the isoelectric point is determined from the detection time of the analyte peak. In the experiment in FIG. 2, each peak of cetuximab (C3-C9) and the peaks of two edge markers and two flanking markers are separated. The advantage of using edge markers is that they can be used in most cases without overlapping with the analyte peak, even when the isoelectric point of the analyte is unknown. On the other hand, when the isoelectric point of the analyte is roughly known, it is possible to select flanking markers, which is expected to improve the precision of determination of isoelectric point, since a pH gradient formed by carrier ampholytes is not necessarily linear.

On the other hand, peptides labeled with dyes that fluoresce in the visible region can also be used as pI markers. In this case, detection of the pI markers requires detection of fluorescence in the visible region, but if the pI markers do not contain tryptophan, it becomes possible to detect the analyte cetuximab and the pI markers independently. In other words, the markers do not affect the separation trace of the analyte, and the analyte does not affect the separation trace of the markers. As a result, even if many pI markers are added to the sample, the signals derived from the analyte and the signals derived from the marker do not become contaminated, and even for a analyte with an unknown isoelectric point, it becomes possible to appropriately select the most desirable pair of pI markers that are closest to the analyte peak and determine the isoelectric point of the analyte more precisely.

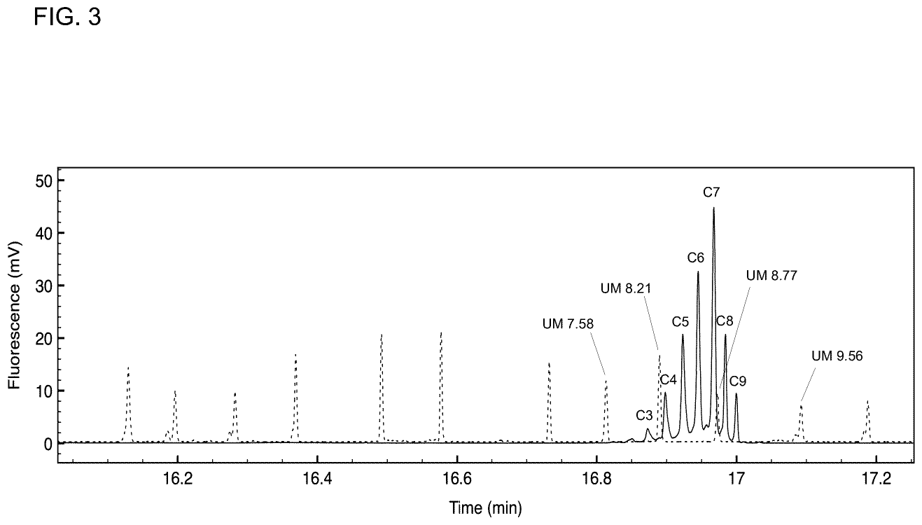

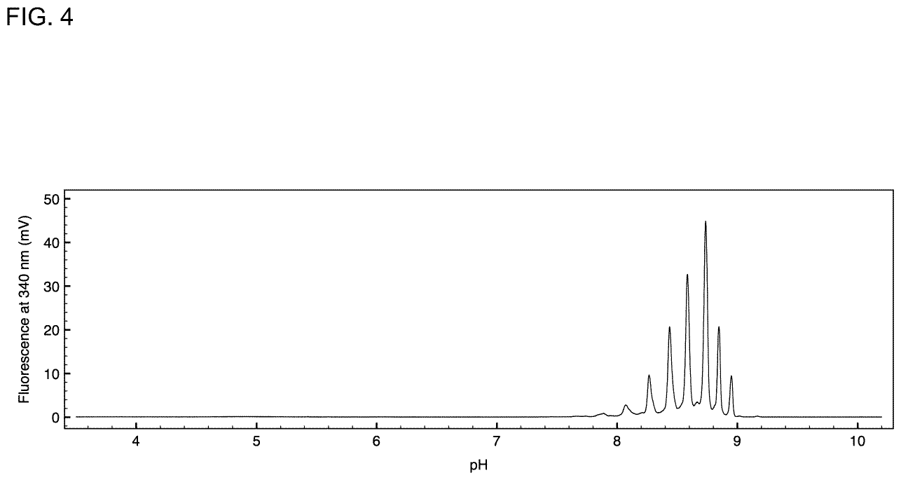

FIG. 3 shows the results of adding twelve kinds of pI markers as ubiquitous markers (UM), which are tryptophan-free peptides labeled with tetramethylrhodamine, to cetuximab and separating them by CIEF. Irradiating with 280 nm ultraviolet light, cetuximab was detected by fluorescence at 340 nm (solid line) and rhodamine-labeled pI markers were detected by fluorescence at 575 nm (dotted line). As is clear from the figure, there is no mutual interference between the analyte signal and the marker signal. FIG. 4 shows the separation trace of cetuximab on pH axis. The trace was produced by converting the time axis of FIG. 3 to pH axis based on the detection time of the twelve ubiquitous markers.

### 1.4 Determination of Isoelectric Points Cetuximab Peaks

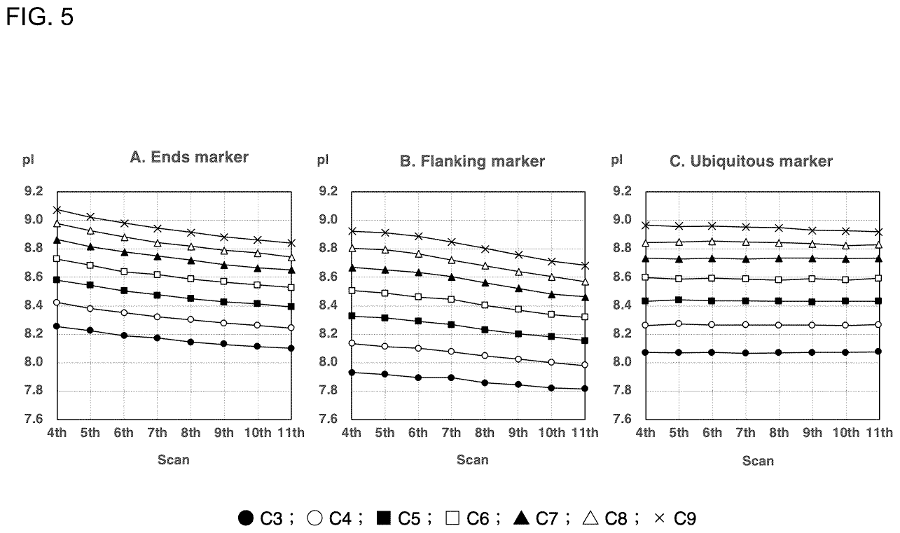

Next, the isoelectric points of the seven main peaks C3 to C9 of cetuximab were determined for each separation result obtained from the third to eleventh scanning detection cycle after the start of focusing at 10 kV using the edge markers (EM), flanking markers (FM), and ubiquitous markers (UM). The isoelectric points were determined based on the assumption that there is a proportional relationship between the isoelectric point and the detection time between the two markers located at both sides of an analyte peak. In the case of the edge marker and flanking marker, all peaks of cetuximab were determined based on the detection time of each set of the two markers, while in the case of the ubiquitous marker, peak C3 was determined based on the detection time of UM7.58 and UM8.21, peaks C4 to C7 were determined based on the detection time of UM8.21 and UM8.77, and peaks C8 and C9 were determined based on the detection time of UM8.77 and UM9.56. The isoelectric point (pI) values of each peak were summarized by the type of marker used and are shown in Table 2. FIG. 5 shows plots of the isoelectric points of the peaks for each scanning detection cycle.

TABLE 2
The isoelectric points of the Cetuximab peaks C3 to C9 determined using
three types of markers for each scan detection cycle from 4th to 11th.

| | | | | | | | | | | | |
|---|---|---|---|---|---|---|---|---|---|---|---|
| 4th | 5th | 6th | 7th | 8th | 9th | 10th | 11th | Average | SD | CV % | A (11th − 4th) |

Edge marker

| | | | | | | | | | | | | |
|---|---|---|---|---|---|---|---|---|---|---|---|---|
| C3 | 8.25 | 8.22 | 8.19 | 8.18 | 8.14 | 8.13 | 8.11 | 8.10 | 8.17 | 0.054 | 0.66 | −0.15 |
| C4 | 8.42 | 8.38 | 8.35 | 8.32 | 8.30 | 8.28 | 8.26 | 8.24 | 8.32 | 0.062 | 0.75 | −0.18 |
| C5 | 8.58 | 8.54 | 8.50 | 8.48 | 8.45 | 8.43 | 8.42 | 8.39 | 8.47 | 0.066 | 0.78 | −0.19 |
| C6 | 8.73 | 8.68 | 8.64 | 8.62 | 8.59 | 8.57 | 8.55 | 8.53 | 8.61 | 0.069 | 0.80 | −0.20 |
| C7 | 8.87 | 8.82 | 8.78 | 8.75 | 8.72 | 8.69 | 8.67 | 8.65 | 8.74 | 0.075 | 0.86 | −0.21 |
| C8 | 8.98 | 8.93 | 8.88 | 8.84 | 8.82 | 8.79 | 8.77 | 8.74 | 8.84 | 0.081 | 0.91 | −0.23 |
| C9 | 9.08 | 9.02 | 8.98 | 8.94 | 8.92 | 8.89 | 8.86 | 8.84 | 8.94 | 0.081 | 0.91 | −0.24 |

Flanking marker

| | | | | | | | | | | | | |
|---|---|---|---|---|---|---|---|---|---|---|---|---|
| C3 | 7.93 | 7.92 | 7.89 | 7.89 | 7.86 | 7.84 | 7.82 | 7.82 | 7.87 | 0.043 | 0.54 | −0.11 |
| C4 | 8.14 | 8.11 | 8.10 | 8.08 | 8.05 | 8.03 | 8.00 | 7.98 | 8.06 | 0.056 | 0.69 | −0.16 |
| C5 | 8.32 | 8.32 | 8.29 | 8.27 | 8.23 | 8.20 | 8.18 | 8.16 | 8.25 | 0.063 | 0.77 | −0.17 |
| C6 | 8.51 | 8.49 | 8.46 | 8.44 | 8.40 | 8.37 | 8.34 | 8.32 | 8.42 | 0.070 | 0.83 | −0.19 |
| C7 | 8.67 | 8.65 | 8.64 | 8.61 | 8.56 | 8.52 | 8.48 | 8.46 | 8.57 | 0.080 | 0.93 | −0.21 |
| C8 | 8.81 | 8.79 | 8.77 | 8.72 | 8.68 | 8.64 | 8.60 | 8.57 | 8.70 | 0.088 | 1.02 | −0.24 |
| C9 | 8.92 | 8.91 | 8.89 | 8.85 | 8.80 | 8.76 | 8.71 | 8.68 | 8.82 | 0.092 | 1.04 | −0.24 |

Ubiquitous marker

| | | | | | | | | | | | | |
|---|---|---|---|---|---|---|---|---|---|---|---|---|
| C3 | 8.07 | 8.07 | 8.07 | 8.07 | 8.07 | 8.07 | 8.07 | 8.08 | 8.07 | 0.003 | 0.03 | 0.00 |
| C4 | 8.26 | 8.27 | 8.27 | 8.26 | 8.26 | 8.26 | 8.26 | 8.27 | 8.27 | 0.004 | 0.05 | 0.01 |
| C5 | 8.43 | 8.44 | 3.43 | 8.44 | 8.43 | 8.43 | 8.43 | 8.43 | 8.43 | 0.003 | 0.04 | 0.00 |
| C6 | 8.60 | 8.59 | 8.60 | 8.59 | 8.58 | 8.59 | 8.58 | 8.59 | 8.59 | 0.006 | 0.07 | 0.00 |
| C7 | 8.73 | 8.73 | 8.73 | 8.73 | 8.74 | 8.74 | 8.73 | 8.74 | 8.73 | 0.004 | 0.04 | 0.00 |
| C8 | 8.84 | 8.85 | 8.85 | 8.85 | 8.84 | 8.84 | 8.82 | 8.83 | 8.84 | 0.011 | 0.12 | −0.01 |
| C9 | 8.97 | 8.96 | 8.96 | 8.95 | 8.95 | 8.93 | 8.93 | 8.92 | 8.95 | 0.017 | 0.19 | −0.05 |

Average of CV % 0.81
Average of CV % 0.83
Average of CV % 0.08
Note,
SD: standard deviation, CV: coefficient of variation, CV %: SD/Average × 100.

Looking at the edge marker and flanking marker, it can be seen that the determined isoelectric point value gradually decreases as the number of scan cycle increases, i.e., the time under focusing conditions is extended. Looking at the C9 peak, the difference in isoelectric point between the 4th and 11th scans is 0.24 pH units smaller for edge marker and flanking marker. Even in the case of the most acidic C3 peak, where the change is smaller, decreases of 0.15 and 0.11 were observed for each type of marker. The reason why the determined isoelectric point value changes with focusing time in this way is thought to be due to the fact that the deviation from the proportional relationship between the position in the capillary and the pH increases with the extension of the focusing time. Since the time required for focusing can differ depending on the protein analyte, it is problematic that the determined isoelectric point value changes depending on the focusing time. In addition, if a uniformly long focusing time is set, the efficiency of the analysis decreases.

On the other hand, for the ubiquitous marker, the change of the determined pI value with the extension of the focusing time was significantly smaller, less than ±0.01 for C3 to C8, and even for C9, which showed a relatively large change, the decrease was 0.05. The small change in the isoelectric point value determined using the ubiquitous marker is also evident from the average CV value for seven peaks, which represents the variation in the isoelectric point value determined for each peak every eight scans. The average CV value was 0.81% for the edge marker, 0.83% for the flanking marker, and 0.08% for the ubiquitous marker. This shows that by using the ubiquitous marker, the isoelectric point can be determined with high precision regardless of the length of focusing time.

The above results show that the isoelectric point of an analyte can be determined with higher precision, regardless of the focusing time, by using multiple markers that ubiquitously present throughout the pH gradient, compared to determining the isoelectric point of an analyte using two markers at both edge of the pH gradient or on both sides of the analyte. A marker containing tryptophan has the advantage that both the analyte and the marker can be detected at a single fluorescent wavelength. However, when used as a ubiquitous marker, the analyte peak and the marker peak may overlap, making it difficult to identify the peaks. In addition, in an analyte with multiple peaks with different isoelectric points, such as cetuximab in this example, it is necessary to separate only the sample that does not contain the marker in order to determine the composition ratio of each peak.

In contrast, in the case of a marker that does not contain tryptophan, the marker must be detected at a different fluorescence wavelength from the analyte, but since the analyte and the marker can be detected independently, overlapping of the analyte peak and the marker peak is not a problem at all. Therefore, by simply adding a mixture of ubiquitous markers to the sample and performing a single separation, it is possible to simultaneously determine both the isoelectric points and the peak composition ratio with high precision. In addition, the method disclosed herein can determine the isoelectric point with high precision by using the pI marker as an internal standard as described above, so that the analyte can be easily given not only the separation trace under specific analytical conditions, but also the physical property value of the isoelectric point, and the isoelectric point information provided in this way can be commonly used by multiple practitioners who handle the same analyte, regardless of the laboratory conditions, and is therefore highly valuable.

Furthermore, tetramethylrhodamine, which was used as a label of ubiquitous markers in this experiment, has an excitation maximum at approximately 550 nm, but in this example, excitation was performed with light of 280 nm. Although 280 nm illumination light is suitable for the fluorescent detection of tryptophan, it is not the most suitable wavelength for the fluorescent detection of tetramethylrhodamine, but the pI marker could be detected without any problems. It is advantageous to be able to perform measurements using a single light source.

### Example 2: Approximation Formula for Determining pI Value

In Example 1, a linear approximation was performed for adjacent pI markers, but a different approximation may be used. Since the pH gradient in isoelectric focusing is non-linear, a curve approximation may be suitable in some case.

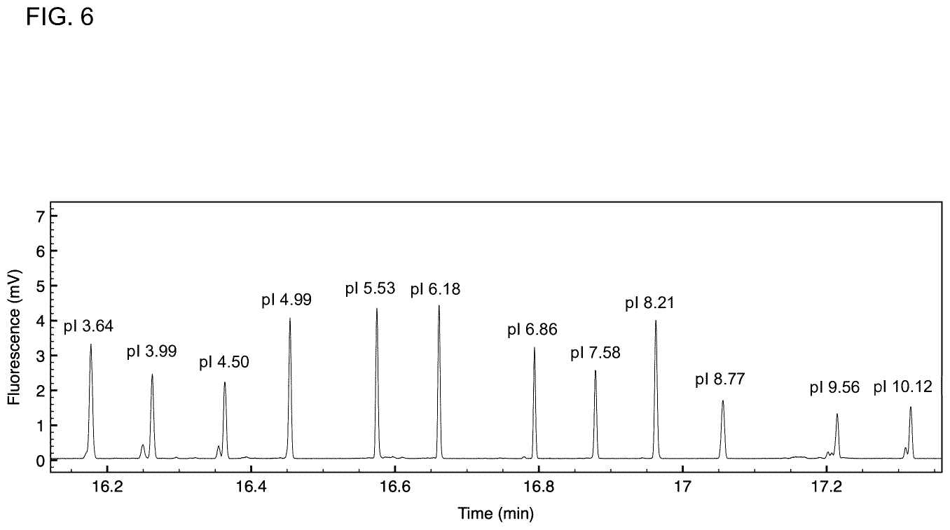

As in Example 1, twelve labelled tryptophan-free pI markers were mixed with the sample and subjected to capillary isoelectric focusing. The results of detecting the pI markers using fluorescence at 575 nm are shown in FIG. 6.

Based on this result, a third-order approximation was performed using the least squares method, and the following equation was obtained:

$$pH = - 2.7 \; 227 \times {t}^{3} + 137.38 \times {t}^{2} - 2303.9 \times t + 12850$$

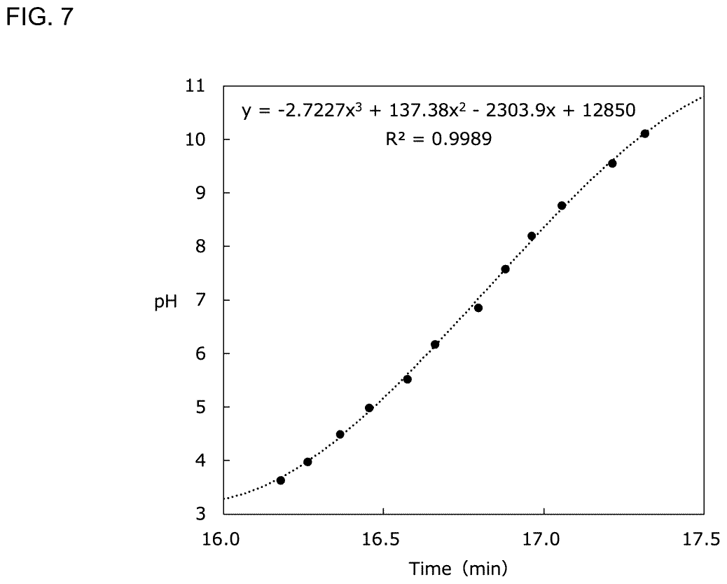

The graph reflecting this is shown in FIG. 7.

For such a more advanced approximation, it is preferable to measure the distribution of many pI markers. Here, an example of a third-order approximation is shown, but it will be understood by those skilled in the art that an approximation of a different order may be used, or an approximation other than a polynomial may be used.

### Note

Although the present disclosure has been illustrated using preferred embodiments thereof, it is understood that the scope of the present invention should be interpreted only by the claims. It is understood that the patents, patent applications, and other documents cited in this specification are incorporated by reference into this specification in the same manner as if the contents themselves were specifically set forth in this specification.

### INDUSTRIAL APPLICABILITY

The present disclosure provides an analysis method by electrophoresis using an internal standard, which can be used in the analysis of various biomolecules such as proteins.

### DESCRIPTION OF SYMBOLS

1: Capillary
2: Excitation light
3: Fluorescence
4: Lens
5: Beam splitter
6 :280 nm bandpass filter
7: 340 nm bandpass filter
8: 575 nm bandpass filter
9: Light source
10: Photodetector

1. 1 A method for measuring an analyte, which is a protein and/or polypeptide, by electrophoresis, comprising the steps of:
subjecting a sample containing the analyte and standards to electrophoresis simultaneously in the same separation space;
detecting the analyte with an optical signal derived from tryptophan and detecting the standards with an optical signal of a different wavelength from the analyte;
and measuring the analyte based on the optical signal.

2. The method of claim 1, further comprising:
determining an isoelectric point of the analyte, wherein the standards have known isoelectric points.

3. The method of claim 1, wherein the standards have different known isoelectric points ranging from 2.5 to 11.5 from one another.

4. The method of claim 1, further comprising:
determining a molecular weight of the analyte, wherein the standards have known molecular weights.

5. The method of claim 4, wherein the standards have different known molecular weights ranging from 5 kDa to 1000 kDa from one another.

6. The method according to claim 1, further comprising deriving an equation expressing a correlation between an isoelectric point and a detection time or detection position, or between a molecular weight and a detection time or detection position, based on optical signals derived from a plurality of standard substances.

7. The method of claim 6, further comprising converting the detection time or detection position of an electropherogram into a pH value or a molecular weight based on the equation.

8. The method of claim 1, wherein each of the standards is a peptide or protein that does not contain tryptophan.

9. The method of claim 1, wherein each of the standards is a dye-labeled peptide or protein.

10. The method of claim 1, wherein each of the standards has a chromophore, fluorophore, or dye label.

11. The method of claim 1, wherein the step of detecting the optical signal comprises irradiating with light having two wavelengths, a wavelength of 280 nm and a wavelength suitable for detecting the chromophore, fluorophore or dye label of each of the standards.

12. The method of claim 1, wherein each of the standards is labeled with rhodamine.

13. The method of claim 1, wherein the step of detecting the optical signal comprises illuminating with light having a single wavelength.

14. The method of claim 13, wherein the single wavelength is 280 nm.

15. The method of claim 1, wherein the standards are prepared so as to be detected as peaks of distinguishable different magnitudes.

16. The method of claim 1, wherein the electrophoresis is capillary electrophoresis.

17. An apparatus for measuring an analyte by electrophoresis, comprising:
a flow path and electrodes for electrophoresis;
a light source for illuminating a portion of the flow path with light; and
a photodetector for receiving light from the flow path,
wherein the photodetector is configured to detect light having a wavelength of 350 nm and light having a wavelength different from 350 nm.

18. The apparatus of claim 17, wherein the wavelength different from 350 nm is 575 nm.

19. A method for producing a standard substance for determining the molecular weight of an analyte, comprising the steps of:
identifying a gene encoding a protein having a known molecular weight;
identifying the base sequence encoding all tryptophan residues of the protein in the gene;
obtaining a modified gene by replacing the portion encoding all tryptophan residues with a base sequence encoding amino acids other than tryptophan;
obtaining a protein produced using the modified gene;
and obtaining a conjugate formed by binding a labeling dye to the protein as the standard substance.
